(19) United States
(12) Patent Application Publication
(10) Pub. No.: US 2022/0287874 A1
(43) Pub. Date: Sep. 15, 2022

(54) Methods, Systems and Apparatus for Direct Connection of Gel Pad Targeted Temperature Management Device

(71) Applicant: C. R. Bard, Inc., Franklin Lakes, NJ (US)

(72) Inventors: Ping Huang, Covington, GA (US); Patrick Hudson Chancy, Atlanta, GA (US); Zhihui Yin, Lilburn, GA (US); Hannah Rose Kriscovich, Marietta, GA (US); Kevin A. Luczynski, Atlanta, GA (US); Sean E. Walker, Platteville, CO (US)

(21) Appl. No.: 17/686,301

(22) Filed: Mar. 3, 2022

Related U.S. Application Data
(60) Provisional application No. 63/158,361, filed on Mar. 9, 2021.

(51) Int. Cl. A61F 7/00

(52) U.S. Cl. CPC A61F 7/0085; A61F 2007/0054; A61F 2007/0056

(57) ABSTRACT

A targeted temperature management (TTM) system for providing a TTM fluid including a thermal pad is disclosed. The pad includes a fluid delivery conduit and a fluid return conduit extending continuously from the pad to the TTM module. Valves may be included in line with fluid delivery and fluid return conduits. The thermal pads include one or more conduit retention devices for binding the fluid conduits together. Methods of using the system are disclosed and include disposing a thermal pad in a storage configuring where the fluid conduits are wound together to form a coil, winding the fluid conduits together to form a coil, binding the windings together with a conduit retention device, and placing the coil underneath a stretchable band of the thermal pad.

### PRIORITY

This application claims the benefit of priority to U.S. Provisional Application No. 63/158,361, filed Mar. 8, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled with a patient to affect surface-to-surface thermal energy exchange with the patient. In general, TTM systems comprise a TTM fluid control module coupled with at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain thermally intimate pad-to-patient contact is of importance to fully realizing medical efficacies with TTM systems.

A fluid delivery line generally includes at least two fluid conduits for transporting TTM fluid to and from the thermal pad. In some instances of TTM therapy, two or more thermal pads may be used. To facilitate transportation of the TTM fluid to and from more than one thermal pad, the fluid delivery line generally incudes a manifold. A fluid delivery line generally includes a proximal portion having two fluid conduits extending distally from the TTM module to the manifold and a distal portion having two fluid conduits for each thermal pad extending distally from the manifold to the thermal pads. As such, the manifold is configured for coupling of multiple pairs of fluid conduits thereto. When setting up a TTM system for performing a TTM therapy utilizing multiple thermal pads, the clinician must connect multiple fluid conduits to the manifold. In some instances, a fluid conduit may not be fully connected to the manifold resulting in a leak. Since in some instances up to about 12 fluid conduits may be connected to the manifold, the occurrence rate of at least a single misconnection resulting in leak may be significant enough to cause concern for the facility and the patient. Furthermore, the manifold may be located in close proximity to the patient exposing the patient to leaked TTM fluid. Disclosed herein are embodiments of systems, devices, and methods for eliminating the possibility of leaks of a TTM fluid when performing the TTM therapy.

During the TTM therapeutic procedure, the therapy may need to be temporarily suspended for the performance of additional medical procedures or other interrupting circumstances. In such instances, the thermal pads may be removed from the patient. While the pads are separated from the patient, there is a need to temporarily store the pads and the associated fluid conduits at locations that allow for movement of the patient and/or the performance of the other medical procedures. Disclosed herein are embodiments of systems, devices, and methods for disposing the thermal pads and fluid conduits in a temporary storage configuration.

### SUMMARY OF THE INVENTION

Briefly summarized, disclosed herein is a targeted temperature management (TTM) system. The system includes a TTM module configured to provide a TTM fluid and a thermal pad configured to facilitate thermal energy transfer between the TTM fluid and a patient.

The pad includes a fluid delivery conduit extending continuously from the pad to the TTM module, where the fluid delivery conduit is configured to facilitate TTM fluid flow from the TTM module to the pad. The pad also includes a fluid return conduit extending continuously from the pad to the TTM module, where the fluid return conduit is configured to facilitate return flow of the TTM fluid from the pad to the TTM module.

The system further includes a valve disposed in line with the fluid delivery conduit, where the valve is configured to selectively allow and prevent flow of TTM fluid through the fluid delivery conduit to the pad.

In some embodiments, the valve is configured to automatically allow TTM fluid flow through the fluid delivery conduit upon connection of the fluid delivery conduit with the TTM module and prevent TTM fluid flow through the fluid delivery conduit upon disconnection of the fluid delivery conduit from the TTM module.

In other embodiments, the valve is configured for actuation by a processor of the TTM module in accordance with a flow control logic of the TTM module to selectively allow and prevent TTM fluid flow through the fluid delivery conduit.

The fluid delivery conduit may be coupled with the TTM module via a first type of connector (referred to herein as an “A-type connector”) attached to the fluid delivery conduit and a second type of connector (referred to herein as a “B-type connector”) attached to the TTM module. Similarly, the fluid return conduit may be coupled with the TTM module via a B-type connector attached to the fluid return conduit and an A-type connector attached to the TTM module. Each A-type connector may be configured to couple with and only with a B-type connector, and each B-type connector may be configured to couple with and only with an A-type connector.

In some embodiments, each A-type connector includes a valve configured to automatically allow TTM fluid flow through the A-type connector upon connection of the A-type connector with a B-type connector and automatically prevent TTM fluid flow through the A-type connector upon disconnection of the A-type connector from the B-type connector. Similarly, each B-type connector may include a valve configured to automatically allow TTM fluid flow through the B-type connector upon connection of the B-type connector with an A-type connector and automatically prevent TTM fluid flow through the B-type connector upon disconnection of the B-type connector from the A-type connector.

The system may include two or more conduit retention devices disposed along the fluid delivery conduit and/or the fluid return conduit, where each conduit retention device is configured for binding at least the fluid delivery conduit and the fluid return conduit together. The thermal pad may include at least one of the one or more conduit retention devices.

The conduit retention device may include a loop and the loop may be threaded onto the fluid delivery conduit or the fluid return conduit. In some embodiments, the loop is threaded onto the fluid delivery conduit together with the fluid return conduit.

The thermal pad may include a stretchable band extending across a top side of the thermal pad, where the stretchable band is configured for disposing the thermal pad in a storage configuration. The storage configuration may include one or both of the fluid delivery conduit and the fluid return conduit disposed in a coiled configuration and may further include placement of the coiled configuration between the stretchable band and the top side.

The thermal pad may include a filter coupled to a fluid containing layer of the pad so that TTM fluid circulating through the fluid containing layer passes through the filter. The filter may include a porous wall oriented parallel to a continuous flow path through the filter.

Further disclosed herein is a medical pad for exchanging thermal energy between a targeted temperature management (TTM) fluid and a patient. The pad includes a fluid containing layer, where the fluid containing layer is configured for containing the TTM fluid. The fluid containing layer comprises a fluid inlet and a fluid outlet, and the TTM fluid is circulatable within the fluid containing layer from the fluid inlet to the fluid outlet. The pad further includes a fluid delivery conduit having a distal end coupled to the fluid inlet, and the fluid delivery conduit is configured to extend continuously from the fluid containing layer to a TTM module. The fluid delivery conduit further includes a first A-type connector at a proximal end, where the first A-type connector is configured to couple with a first B-type connector disposed on a connection panel of the TTM module. The pad further includes a fluid return conduit having a distal end coupled to the fluid outlet and the fluid return conduit is configured to extend from the fluid containing layer to the TTM module. The fluid return conduit also includes a second B-type connector at a proximal end, where the second B-type connector is configured to couple with a second A-type connector disposed on the connection panel of the TTM module. In some embodiments, each A-type connector is configured to couple with and only with a B-type connector, and each B-type connector is configured to couple with and only with an A-type connector.

Each A-type connector may include a valve configured to automatically allow TTM fluid flow through the A-type connector upon connection of the A-type connector with a B-type connector and automatically prevent TTM fluid flow through the A-type connector upon disconnection of the A-type connector from the B-type connector. Similarly, each B-type connector may include a valve configured to automatically allow TTM fluid flow through the B-type connector upon connection of the B-type connector with the A-type connector and automatically prevent TTM fluid flow through the B-type connector upon disconnection of the B-type connector from the A-type connector.

The pad may further include a conduit retention device configured to bind at least the fluid delivery conduit and the fluid return conduit together. The conduit retention device may include a loop, and the loop may be threaded onto at least one of the fluid delivery conduit or the fluid return conduit. The loop may also be threaded onto the fluid delivery conduit and the fluid return conduit.

The pad may further include a stretchable band extending across a top side of the pad, where the stretchable band is configured for disposing the pad in a storage configuration. The storage configuration may include one or both of the fluid delivery conduit and the fluid return conduit disposed in a coiled configuration and placement of the coiled configuration between the stretchable band and the top side.

The pad may further include a filter coupled to the fluid containing layer so that TTM fluid circulating through the fluid containing layer passes through the filter and the filter may include a porous wall disposed parallel to a continuous flow path through the filter.

Also disclosed herein is a method of using a targeted temperature management (TTM) system to exchange thermal energy with a patient. The method includes providing a TTM module configured to circulate TTM fluid through one or more thermal pads. The method also includes providing a first thermal pad, where the first thermal pad includes a first pad portion configured for placement on the patient. The first thermal pad further includes a first fluid delivery conduit coupled to the first pad portion and a first fluid return conduit coupled to the first pad portion.

The method further includes applying the first pad portion to the patient. The method also includes extending the first fluid delivery conduit from the first pad portion to the TTM module, and connecting the first fluid delivery conduit to a connection panel of the TTM module. The method also includes extending the first fluid return conduit from the first pad portion to the TTM module, and connecting the first fluid return conduit to the connection panel of the TTM module. The method also includes circulating TTM fluid through the first thermal pad.

In some embodiments, the first thermal pad includes a first conduit retention device coupled to one of the first fluid delivery conduit or the first fluid return conduit, and the method further includes binding the first fluid delivery conduit and the first fluid return conduit together via the first conduit retention device.

In some embodiments, the method includes providing a second thermal pad including a second pad portion configured for placement on the patient, a second fluid delivery conduit coupled to the second pad portion, a second fluid return conduit coupled to the second pad portion, and a second conduit retention device coupled to one of the second fluid delivery conduit or the second fluid return conduit. The method may further include applying the second pad portion to the patient. The method may also include extending the second fluid delivery conduit from the second pad portion to the TTM module and connecting the second fluid delivery conduit to a connection panel of the TTM module. The method may also include extending the second fluid return conduit from the second pad portion to the TTM module and connecting the second fluid return conduit to the connection panel of the TTM module. The method may also include circulating TTM fluid through the second thermal pad.

The method may further include binding three or more of the first fluid delivery conduit, the first fluid return conduit, the second fluid delivery conduit, and the second fluid return conduit together via the first conduit retention device, and the method may further include binding two or more of the first fluid delivery conduit, the first fluid return conduit, the second fluid delivery conduit, and the second fluid return conduit together via the second conduit retention device.

The method may further include removing the first pad portion from the patient, disconnecting the first fluid delivery conduit from the connection panel of the TTM module, disconnecting the first fluid return conduit from the connection panel of the TTM module, winding the first fluid delivery conduit together with the first fluid return conduit to form a coil, and binding the windings of the coil together via the first conduit retention device to maintain the coil.

In some embodiments of the method, the first thermal pad includes a stretchable band extending across a top side of the first pad portion, and the method further includes placing the coil between the band and the top side to dispose the thermal pad in a storage configuration. The method may further include binding a bedrail together with the windings of the coil via the first conduit retention device to couple the first thermal pad to the bedrail.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a current embodiment of a targeted temperature management (TTM) system for cooling or warming a patient showing fluid delivery lines extending away from a TTM module.

FIG. 1B illustrates a back side of the TTM module of FIG. 2A showing the connection of the fluid delivery lines to the TTM module.

FIG. 2A illustrates a targeted temperature management (TTM) system having fluid conduits extending continuously from the thermal pads to the TTM module, in accordance with some embodiments.

FIG. 2B illustrates a connection panel on a back side of the TTM module of FIG. 2A, in accordance with some embodiments.

FIG. 3A illustrates a hydraulic schematic of the TTM system of FIG. 2A, in accordance with some embodiments.

FIG. 3B illustrates a block diagram depicting various elements of a console of the TTM module of FIG. 2A, in accordance with some embodiments.

FIG. 4A is a top view of the thermal pad of FIG. 2A, in accordance with some embodiments.

FIG. 4B is a cross-sectional view of the pad of FIG. 4A cut along sectioning lines 4B-4B, in accordance with some embodiments.

FIG. 4C is a top view of the thermal pad of FIG. 4A disposed in a storage configuration, in accordance with some embodiments.

FIG. 5A is a front perspective view of the conduit retention device of FIG. 2A, in accordance with some embodiments.

FIG. 5B illustrates a use case of the conduit retention device of FIG. 5A, in accordance with some embodiments.

FIG. 5C is a cross-sectional view of the conduit retention device as employed in the use case of FIG. 5B, in accordance with some embodiments.

FIG. 6A provides an exploded perspective view of a TTM fluid filter, in accordance with some embodiments.

FIG. 6B is a cross-sectional side view of the filter of FIG. 6A, in accordance with some embodiments.

FIG. 6C is a cross-sectional detail view of the thermal contact pad of FIG. 4A incorporating the filter of FIG. 6A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “horizontal,” “vertical” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled with” refer to any form of interaction between two or more entities, including mechanical, electrical, magnetic, electromagnetic, fluid, signal, communicative (including wireless), and thermal interaction. Two components may be connected to or coupled with each other even though they are not in direct contact with each other. For example, two components may be coupled with each other through an intermediate component.

The directional terms “proximal” and “distal” are used herein to refer to opposite locations on a medical device. The proximal end of the device is defined as the end of the device closest to the end-user when the device is in use by the end-user. The distal end is the end opposite the proximal end, along the longitudinal direction of the device, or the end furthest from the end-user.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

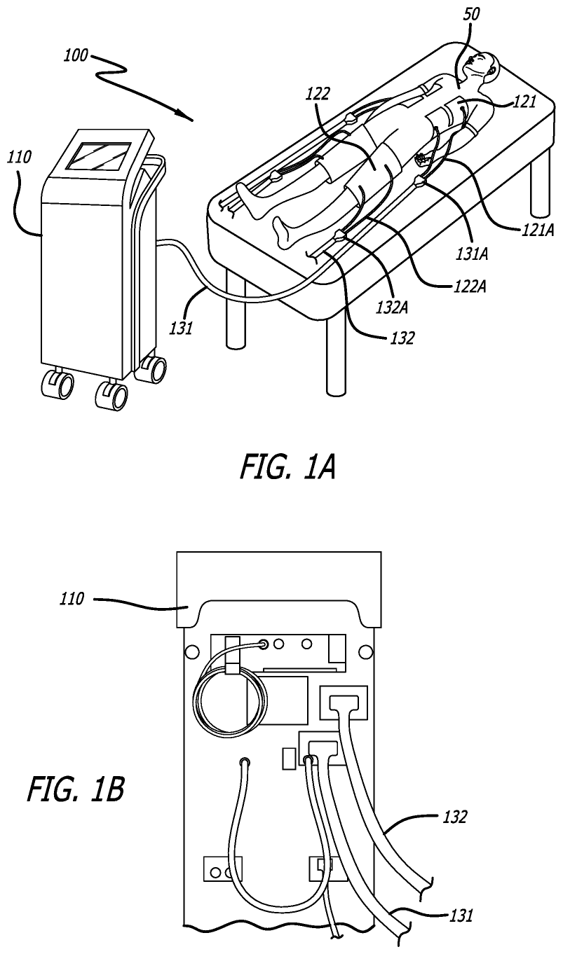

FIGS. 1A and 1B illustrate a current embodiment of a targeted temperature management (TTM) 100. FIG. 1A shows the TTM system 100 having two thermal contact pads 121, 122 coupled to a patient 50 for administering TTM therapy to the patient 50. A TTM module 110 prepares TTM fluid for circulation through the pads 121, 122. The pad 121 includes a pair of fluid conduits 121A coupled to a fluid deliver line 131 via a connection hub 131A. Similarly, pad 122 includes a pair of fluid conduits 122A coupled to a fluid deliver line 132 via a connection hub 132A. The fluid delivery lines 131, 132 provide for TTM fluid communication between the pads 121, 122 and the TTM module 110.

FIG. 1B illustrates a backside of the TTM module of FIG. 1A showing the connection of the fluid delivery lines 131, 132 to the TTM module 110. As illustrated in FIGS. 1A and 1B, the fluid conduits 121A, 122A extend from the pads 121, 122 partially toward the TTM module 110, and the fluid delivery lines 131, 132 extend from the TTM module 110 partially toward the pads 121, 122. The connection hubs 131A, 131B serve as an intermediate fluid connection point between the fluid conduits 121A, 122A and the fluid delivery lines 131, 132.

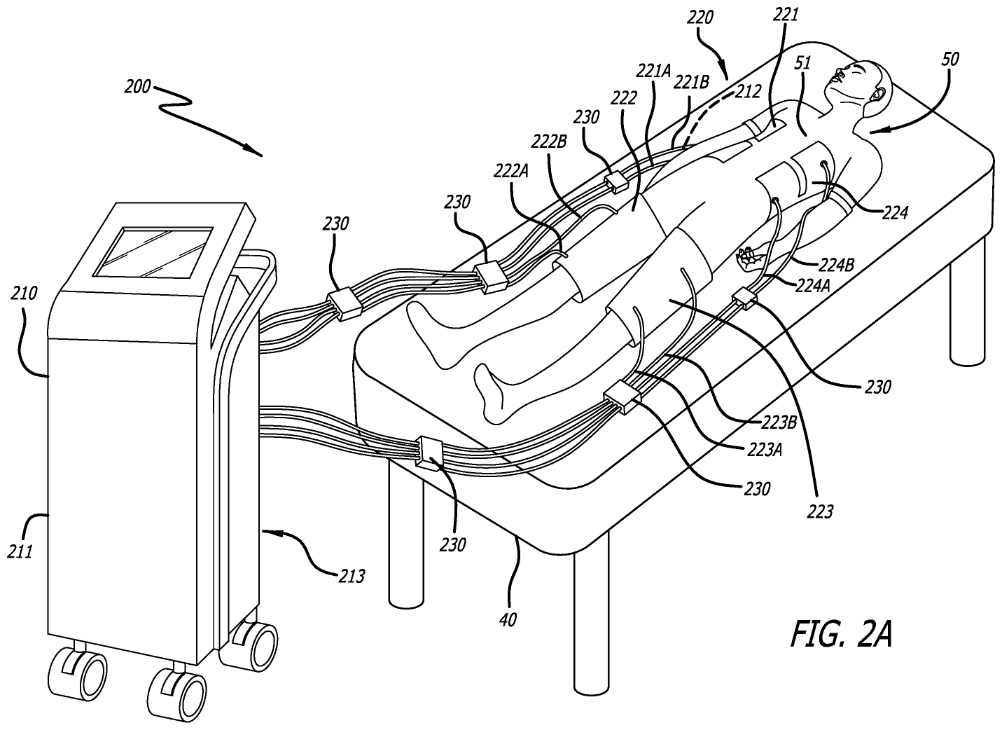

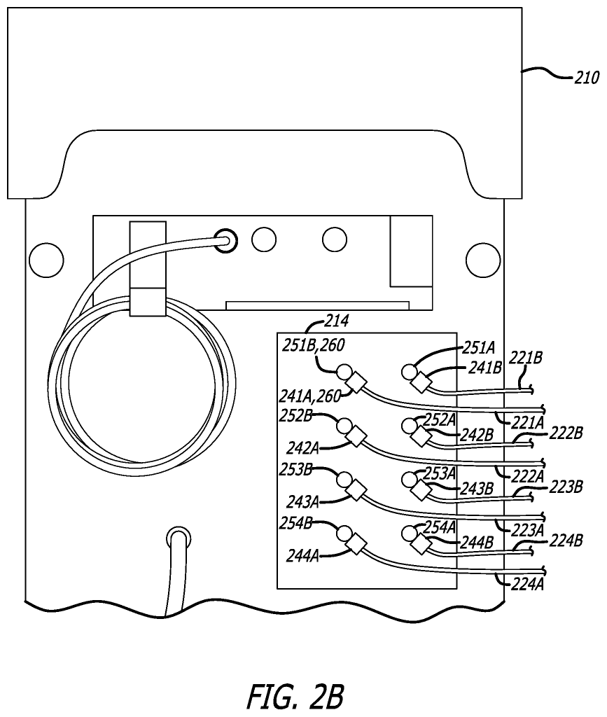

FIG. 2A illustrates a targeted temperature management (TTM) system 200 connected to the patient 50 for administering TTM therapy to the patient 50 which may include a cooling and/or warming of the patient 50 who may be lying on a bed 40, in accordance with some embodiments. The TTM system 200 includes a TTM module 210 and a thermal contact pad set 220. In the illustrated embodiment, the pad set 220 includes four thermal contact pads (pads) 221, 222, 223, and 224. In other embodiments, the pad set 220 may include 1, 2, 3, 5, 6, or more thermal contact pads. Each pad includes two fluid conduits extending to and coupled with the TTM module 210 to facilitate circulating flow of TTM fluid 212 between the pad and the TTM module 210. The pad 221 includes a delivery conduit 221A and a return conduit 221B. Each of the other pads 222-224 includes a delivery conduit and a return conduit designated in the same manner of reference numbering. The delivery conduit 221A and a return conduit 221B extend continuously from the TTM module 210 to the pad 221 to provide for flow of TTM fluid 212 between the TTM module 210 and the pad 221. The system 200 includes one or more conduit retention devices 230 as described in further detail below.

In use, the TTM module 210 prepares the TTM fluid 212 for delivery to the pad set 220 by heating or cooling the TTM fluid 212 to a defined temperature in accordance with a prescribed TTM therapy. The TTM module 210 circulates the TTM fluid 212 between the TTM module 210 and the pad set 220. The pad set 220 is applied to the skin 51 of the patient to facilitate thermal energy exchange between the pad set 220 and the patient 50. During the TTM therapy, the TTM module 210 may continually control the temperature of the TTM fluid 212 toward a target TTM temperature.

In some embodiments, each corresponding pair of fluid conduits, such as the fluid delivery conduit 221A and the fluid return conduit 221B, may be attached together along a length of the fluid conduits. More specifically, the pair of fluid conduits may be attached together along a central portion of the length while allowing separation of the fluid conduits at each end. In some embodiments, the fluid conduits may include color coding or indica (not shown) to indicate a direction of flow of the TTM fluid 212.

FIG. 2B illustrates a connection panel 214 which may be disposed on a backside 213 of the TTM module 210 showing fluid conduit connectors coupled with panel connectors. The conduit delivery connectors 241A-244A are coupled with the fluid delivery conduits 221A-224A, respectively, and the conduit return connectors 241B-244B are coupled to the fluid return conduits 221B-224B, respectively. The connection panel 214 includes panel delivery connectors 251B-154B and panel return connectors 251A-254A. The panel delivery connectors 251B-154B are coupled with the conduit delivery connectors 241A-244A, respectively, and the panel return connectors 251A-254A are coupled with conduit return 241B-244B, respectively. The connectors designated as A-type connectors (i.e., designated by a reference number having an “A” suffix) may functionally correspond with (i.e., couple with) the connectors designated as B-type connectors (i.e., designated by a reference number having an “B” suffix). In some embodiments, an A-type connector may only couple with a B-type connector.

The conduit delivery connector 241A may include a valve 260 which may be integrated into the conduit delivery connector 241A. The valve 260 may be actuated in conjunction with the connecting process of the connector. For example, the valve 260 integrated into the conduit delivery connector 241A may be closed to prevent flow of TTM fluid 212 through the conduit delivery connector 241A unless a corresponding connector (e.g., the panel delivery connector 251B) is coupled thereto. Similarly, the valve 260 may be open to allow flow of TTM fluid 212 through the conduit delivery connector 241A when the corresponding connector is coupled thereto. For example, flow of TTM fluid 212 through the conduit delivery connector 241A is automatically allowed when the panel delivery connector 251B is coupled with the conduit delivery connector 241A and automatically disallowed when a panel delivery connector 251B is decoupled from (or not coupled with) the conduit delivery connector 241A. The automatic nature of the valve 260 may minimize spillage of TTM fluid 212 during connection and disconnection.

In similar fashion, the panel delivery connector 251B may also include a valve 260 which may be integrated into the panel delivery connector 251B. The valve 260 may be actuated in conjunction with the connecting process of the connector. For example, the valve 260 integrated into the panel delivery connector 251B may be closed to prevent flow of TTM fluid 212 through the panel delivery connector 251B unless a corresponding connector (e.g., the conduit delivery connector 241A) is coupled thereto. Similarly, the valve 260 may be open to allow flow of TTM fluid 212 through the panel delivery connector 251B when the corresponding connector is coupled thereto. For example, flow of TTM fluid 212 through the panel delivery connector 251B is automatically allowed when the conduit delivery connector 241A is coupled with the panel delivery connector 251B and automatically disallowed when a conduit delivery connector 241A is decoupled from (or not coupled with) the panel delivery connector 251B.

Although not specifically shown with a reference number, each of the conduit return connectors 241B-244B, panel delivery connectors 251B-254B, and the panel return connectors 251A-254A may include a valve 260. In some embodiments, the valve 260 may be omitted from one or more of the conduit delivery connectors 241A-244A, the conduit return connectors 241B-244B, the panel delivery connectors 251B-254B, and the panel return connectors 251A-254A.

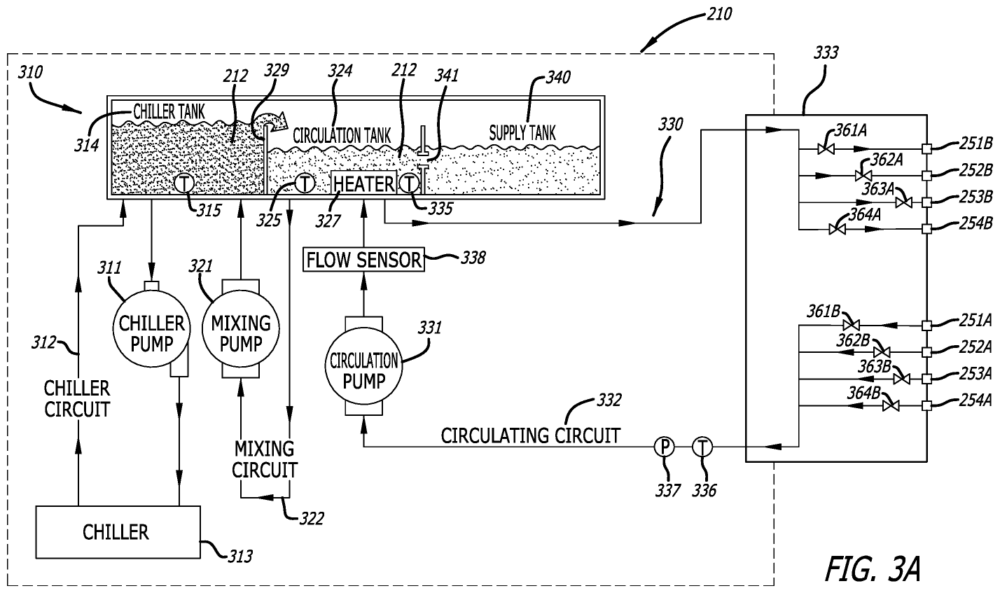

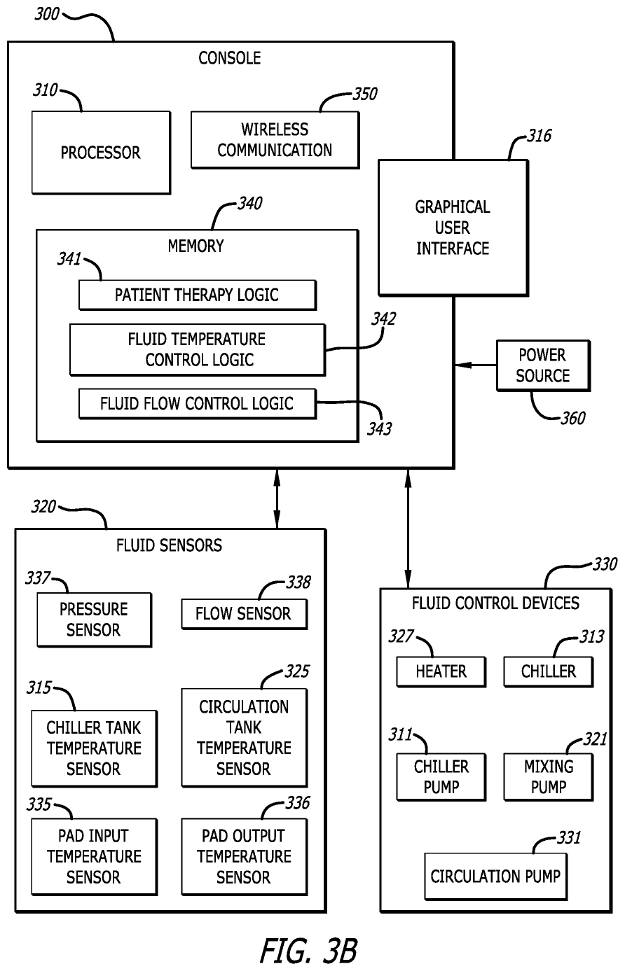

FIG. 3A illustrates a hydraulic schematic of the TTM system 200. The pad set 220 (FIG. 2A) along with the corresponding fluid conduits are disposed external to the housing 211 of the TTM module 210. The TTM module includes various fluid sensors and fluid control devices to prepare and circulate the TTM fluid 212. The fluid subsystems of the TTM module may include a temperature control subsystem 310 and a circulation subsystem 330.

The temperature control subsystem 310 may include a chiller pump 311 to pump (recirculate) TTM fluid 212 through a chiller circuit 312 that includes a chiller 313 and a chiller tank 314. A temperature sensor 315 within the chiller tank 314 is configured to measure a temperature of the TTM fluid 212 within the chiller tank 314. The chiller 313 may be controlled by a temperature control logic (see FIG. 3B) as further described below to establish a desired temperature of the TTM fluid 212 within chiller tank 314. In some instances, the temperature of the TTM fluid 212 within the chiller tank 314 may be less than the target temperature for the TTM therapy.

The temperature control subsystem 310 may further include a mixing pump 321 to pump TTM fluid 212 through a mixing circuit 322 that includes the chiller tank 314, a circulation tank 324, and a dam 328 disposed between the chiller tank 314 and circulation tank 324. The TTM fluid 212, when pumped by the mixing pump 321, enters the chiller tank 314 and mixes with the TTM fluid 212 within the chiller tank 314. The mixed TTM fluid 212 within the chiller tank 314 flows over the dam 328 and into the circulation tank 324. In other words, the mixing circuit 322 mixes the TTM fluid 212 within chiller tank 314 with the TTM fluid 212 within circulation tank 324 to cool the TTM fluid 212 within the circulation tank 324. A temperature sensor 325 within the circulation tank 324 measures the temperature of the TTM fluid 212 within the circulation tank 324. The temperature control logic may control the mixing pump 321 in accordance with temperature data from the temperature sensor 325 within the circulation tank 324.

The circulation tank 324 includes a heater 327 to increase to the temperature of the TTM fluid 212 within the circulation tank 324, and the heater 327 may be controlled by the temperature control logic. In summary, the temperature control logic when executed by the processor (see FIG. 3B) may 1) receive temperature data from the temperature sensor 315 within the chiller tank and the temperature sensor 325 within the circulation tank 324 and 2) control the operation of the chiller 313, the chiller pump 311, the heater 327, and mixing pump 322 to establish and maintain the temperature of the TTM fluid 212 within the circulation tank 324 at the target temperature for the TTM therapy.

The circulation subsystem 330 comprises a circulation pump 313 to pull TTM fluid 212 from the circulation tank 324 and through a circulating circuit 332 that includes the pad set 220 located upstream of the circulation pump 313. The circulating circuit 332 also includes a pressure sensor 337 to represent a pressure of the TTM fluid 212 within the pad set 320. The circulating circuit 332 includes a temperature sensor 335 within the circulation tank 324 to represent the temperature of the TTM fluid 212 entering the pad set 220 and a temperature sensor 336 to represent the temperature of the TTM fluid exiting the pad set 220. A flow meter 338 is disposed downstream of the circulation pump 313 to measure the flow rate of TTM fluid 212 through the circulating circuit 332 before the TTM fluid 212 re-enters that the circulation tank 324.

In use, the circulation tank 324, which may be vented to atmosphere, is located below (i.e., at a lower elevation than) the pad set 220 so that a pressure within the pad set 220 is less than atmospheric pressure (i.e., negative) when TTM fluid flow through the circulating circuit 332 is stopped. The pad set 220 is also placed upstream of the circulation pump 331 to further establish a negative pressure within the pad set 220 when the circulation pump 313 is operating. The fluid flow control logic (see FIG. 3B) may control the operation of the circulation pump 313 to establish and maintain a desired negative pressure within the pad set 220. A supply tank 340 provides TTM fluid 212 to the circulation tank 324 via a port 341 to maintain a defined volume of TTM fluid 212 within the circulation tank 324.

The circulation subsystem 330 may include a manifold 333 for circulating TTM fluid through individual pads of the pad set 220. The manifold 333 may valves 361A-364A for controlling flow of TTM fluid 212 to the pad set 220 via the panel delivery connectors 251B-254B and further includes valves 361B-364B for controlling flow of TTM fluid 212 from the pad set 220 via the panel return connectors 251A-254A. The valves 361A-364A and 361B-364B may be electro-mechanical valves providing for actuation of the valve via flow control logic as further described below in relation to FIG. 3B. In some embodiments, one or more of the valves 361A-364A and 361B-364B may be omitted.

FIG. 3B illustrates a block diagram depicting various elements of the TTM module 210 of FIG. 2A, in accordance with some embodiments. The TTM module 210 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 210.

Illustrated in the block diagram of FIG. 3B are fluid sensors 320 as described above in relation to FIG. 3A. Each of the fluid sensors 320 are coupled with the console 300 so that data from the fluid sensors 320 may be utilized in the performance of TTM module operations. Fluid control devices 330 are also illustrated in FIG. 3B as coupled with the console 300. As such, logic modules may control the operation of the fluid control devices 330 as further described below.

The patient therapy logic 341 may receive input from the clinician via a graphical user interface (GUI) 316 to establish operating parameters in accordance with a prescribed TTM therapy. Operating parameters may include a target temperature for the TTM fluid 212 and/or a thermal energy exchange rate which may comprise a time-based target temperature profile. In some embodiments, the fluid temperature control logic 342 may define other fluid temperatures of the TTM fluid 212 within the TTM module 210, such a target temperature for the TTM fluid 212 within the chiller tank 314, for example.

The fluid temperature control logic 342 may perform operations to establish and maintain a temperature of the TTM fluid 212 delivered to the pad set 220 in accordance with the predefined target temperature. One temperature control operation may include chilling the TTM fluid 212 within the chiller tank 314. The fluid temperature control logic 342 may utilize temperature data from the chiller tank temperature sensor 315 to control the operation of the chiller 313 to establish and maintain a temperature of the TTM fluid 212 within the chiller tank 314.

Another temperature control operation may include cooling the TTM fluid 212 within the circulation tank 324. The fluid temperature control logic 342 may utilize temperature data from the circulation tank temperature sensor 325 to control the operation of the mixing pump 321 to decrease the temperature of the TTM fluid 212 within the circulation tank 324 by mixing TTM fluid 212 from the chiller tank 314 with TTM fluid 212 within circulation tank 324.

Still another temperature control operation may include warming the TTM fluid 212 within the circulation tank 324. The fluid temperature control logic 342 may utilize temperature data from the circulation tank temperature sensor 325 to control the operation of the heater 327 to increase the temperature of the TTM fluid 212 within the circulation tank 324.

The fluid flow control logic 343 may control the operation of the circulation pump 331. As a thermal energy exchange rate is at least partially defined by the flow rate of the TTM fluid 212 through the pad set 220, the fluid flow control logic 343 may, in some embodiments, control the operation of the circulation pump 331 in accordance with a defined thermal energy exchange rate for the TTM therapy.

In some embodiments, the fluid flow control logic 343 may control the flow of TTM fluid 212 to individual pads of the pad set 220 via control of the manifold valves 361A-364A and 361B-364B. For example, the fluid flow control logic 343 may selectively open corresponding valves 361A, 361B to circulate TTM fluid 212 through the pad 221 and/or close corresponding valves 361A, 361B to prevent circulation of TTM fluid 212 through the pad 221. In similar fashion, the fluid flow control logic 343 may control any or all valves 361A-364A and 361B-364B to control the circulation of TTM fluid 212 through any or all of the pads of the pad set 220. In some embodiments, the valves 361A-364A and 361B-364B may be configured to partially allow/prevent fluid flow. In such embodiments, the fluid flow control logic 343 may be configured to individually regulate the circulation of TTM fluid 212 through each pad of the pad set 220.

The console 300 may include or be coupled with a wireless communication module 350 to facilitate wireless communication with external devices. A power source 360 provides electrical power to the console 300.

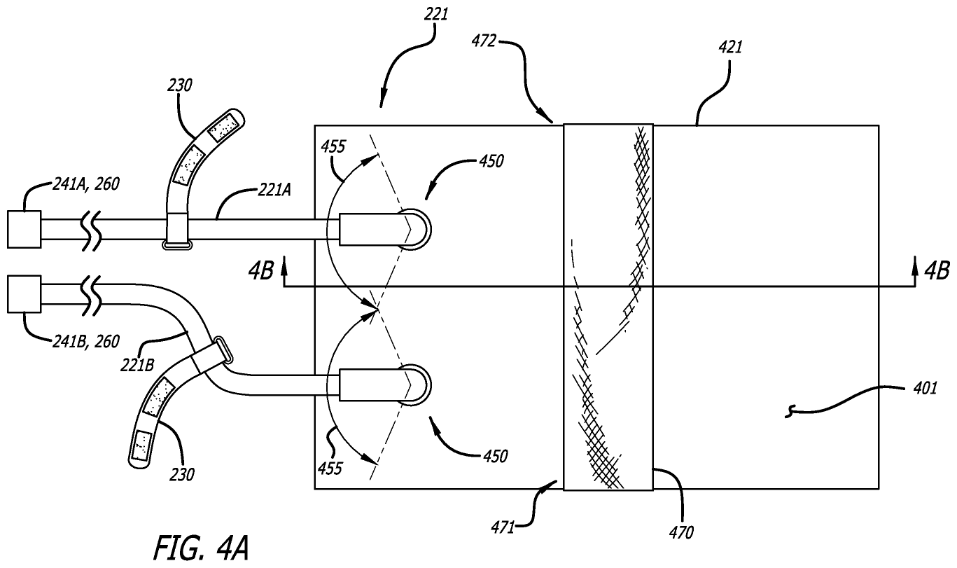

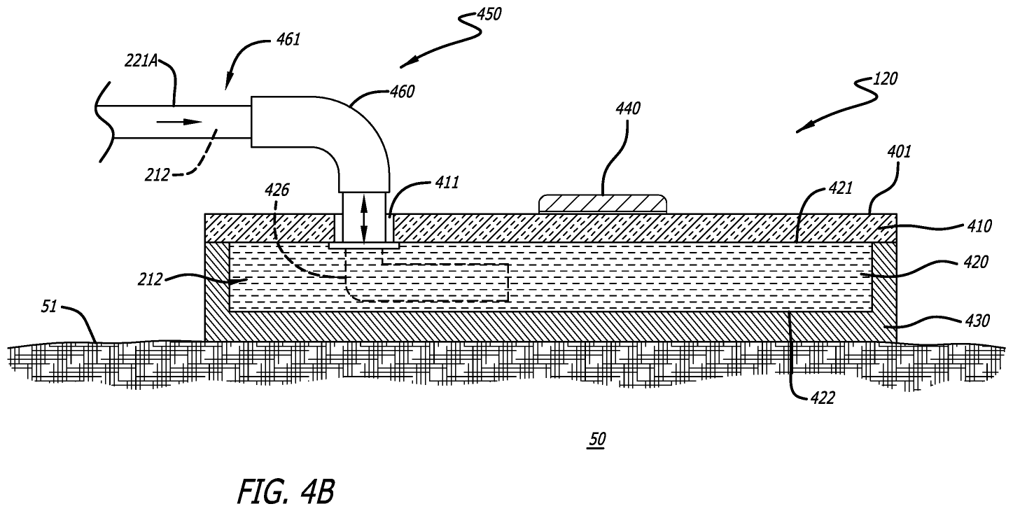

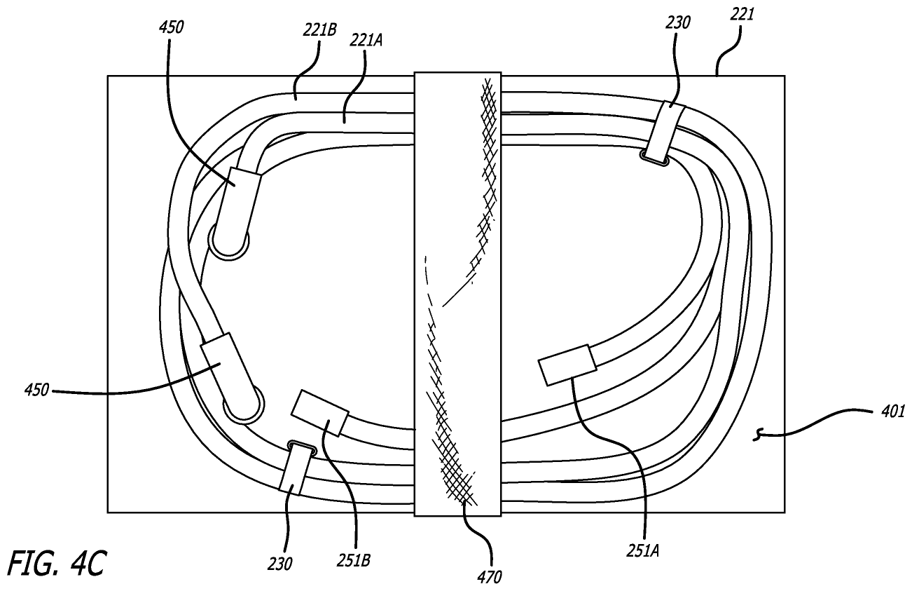

FIG. 4A shows a top view of the thermal contact pad 221. While the description that follows describes features, components, and details of the pad 221, the description that follows may equally apply to any and all other thermal contact pads of the pad set 220 (e.g., pads 222-224). The pad 221 includes the fluid delivery conduit 221A and the fluid return conduit 221B extending away from the joints 450, in accordance with some embodiments. As illustrated, the joints 450 may provide for a rotatable connection of each of the fluid delivery conduit 221A and the fluid return conduit 221B with respect to a pad portion 421 of the pad 221. The rotatable connection may provide for the fluid conduit to rotate through an angle 455 ranging up to about 90 degrees, 180 degrees, 360 degrees, or more. In some embodiments, the joint 450 may define a fixed rotatable connection, i.e., the joint may allow rotation but not separation. In other embodiments, the joint 450 may define a pre-assembled rotatable connection that allows rotation and separation by the clinician. Also shown are the connectors 241A, 241B coupled with the fluid delivery conduit 221A and the fluid return conduit 221B, respectively. As discussed above, each of the connectors 241A, 241B may include a valve 260.

The pad 221 may include a stretchable band 470 extending across a top side of the pad portion 421. As illustrated, the stretchable band 470 extends along a width of the pad portion 421. In other embodiments, the band 470 may extend along a length of the pad portion 421. The band 470 may extend across an entire width of the pad portion 421 or a partial width. The band 470 is attached to the pad portion 421 at the first and second ends 471, 472 of the band 470. In other embodiments, the band 470 may be attached to the pad portion 421 at one or more other locations along the band 470. The band 470 may be formed of any stretchable material, such as a rubber, nylon, cotton having a synthetic or natural rubber core or silicone material that may be cleaned or disinfected according to healthcare facility standards.

The band 470 may be attached to the pad portion 421 in a relaxed state. More specifically, a free length (i.e., the length of the band 470 in a non-stretched state) may be sufficiently long so that the band 470 is in a non-stretched state when the pad portion 421 is applied to the patient 50. In some embodiments, the band 470 may be omitted.

The pad 221 may include one or more conduit retention devices 230 as shown. The conduit retention devices 230 may be coupled to either or both of the fluid delivery conduit 221A and the fluid return conduit 221B.

FIG. 4B shows a cross-sectional side view of the pad portion 421 of the thermal contact pad 221 of FIG. 4A in contact with the patient 50, in accordance with some embodiments. The pad 221 may comprise multiple layers to provide multiple functions of the pad 221. A fluid containing layer 420 is fluidly coupled with the fluid delivery conduit 221A via the joint 450 to facilitate circulation of the TTM fluid 212 within the fluid containing layer 420. Similarly, (although not shown in FIG. 4B) the fluid containing layer 420 is fluidly coupled with the fluid return conduit 221B via the joint 450. The fluid containing layer 420 having TTM fluid 212 circulating therein defines a heat sink or a heat source for the patient 50 in accordance with a temperature of the TTM fluid 212. The fluid delivery conduit 221A may also be coupled with an internal fluid conduit 426 of the fluid containing layer 420 so that TTM fluid 212 entering the fluid containing layer 420 passes through the internal fluid conduit 426.

The pad 221 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence space or air pockets between the thermal conduction layer 430 and the patient 50.

The pad 221 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the fluid delivery conduit 221A and fluid return conduit 221B with the fluid containing layer 420.

The joint 450 may include an elbow 460 to change the orientation of the fluid delivery conduit 221A. As shown, the orientation of the fluid delivery conduit 221A is shifted from an orientation that is perpendicular to the pad 221 to an orientation that is substantially parallel to the pad 221. The elbow 460 also establishes an orientation of a distal portion 461 of the fluid delivery conduit 221A to be substantially parallel to the pad 221 and/or the fluid containing layer 420.

FIG. 4C is a top view of the pad 221 in a storage or shipping configuration, in accordance with some embodiments. As illustrated, the fluid delivery conduit 221A and the fluid return conduit 221B (sometimes referred to herein as the fluid conduits) may be arranged in a coiled configuration (i.e., form a coil) so that the fluid conduits may be disposed between the band 470 and the top side 401 of the pad portion 421. In the illustrated, the band 470 is stretched so that a tension in the band 470 may secure the fluid conduits to the pad portion 421. In some embodiments, the tension may be sufficient to prevent the coiled fluid conduits from separating from the pad portion 421 when the pad 221 is disposed in a vertical orientation. As shown, one or more conduit retention devices 230 may be employed to maintain the fluid conduits in the coiled configuration.

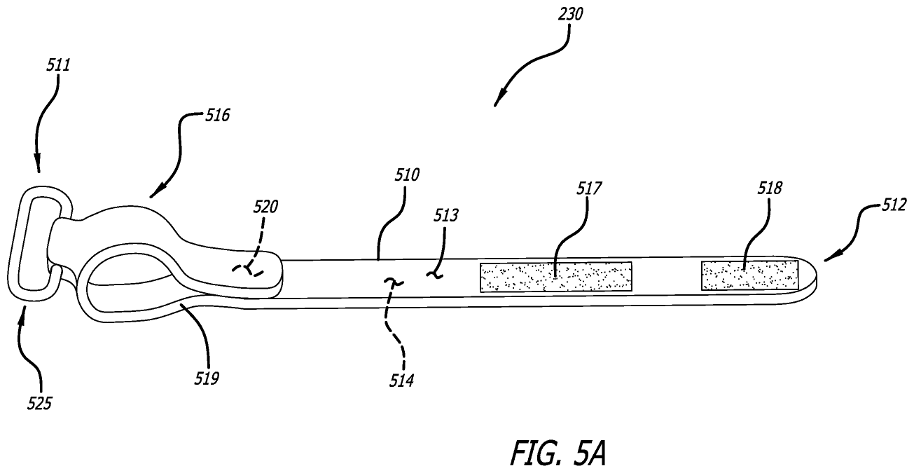

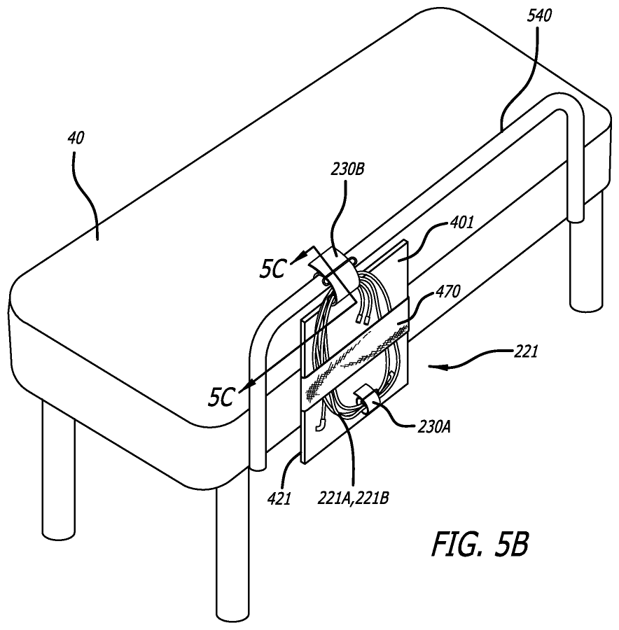

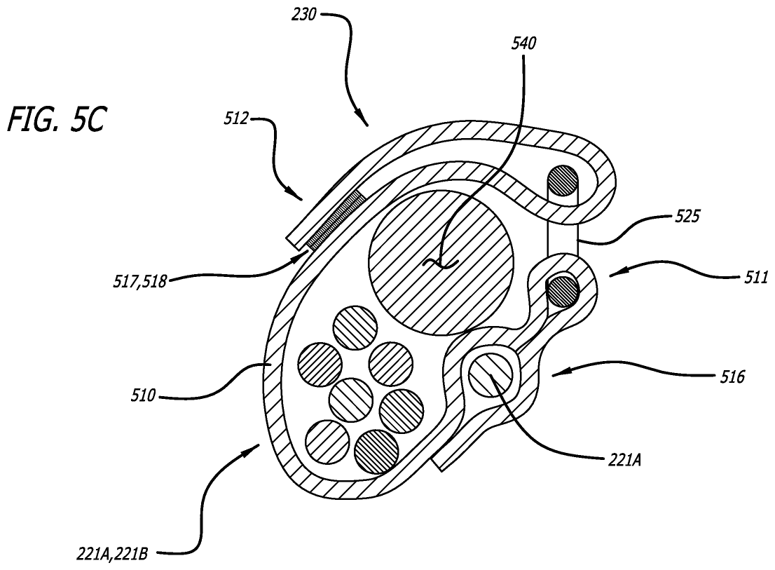

FIG. 5A illustrates the conduit retention device 230 shown in FIG. 2A. As shown in FIG. 2A, the conduit retention device 230 may be configured to couple two on more fluid conduits together. For example, the conduit retention device 230 may couple the fluid delivery conduit 221A with the fluid return conduit 221B. In a further example, the conduit retention device 230 may couple the fluid delivery conduit 221A, the fluid return conduit 221B, the fluid delivery conduit 222A, and the fluid return conduit 222B together. In other words, the conduit retention device 230 attach any of the fluid conduits together.

In some embodiments, the conduit retention device 230 may be pre-attached to a fluid conduit. The conduit retention device 230 may define a sliding attachment so that the conduit retention device 230 may be selectively positioned along a length of the fluid conduit. In other embodiments, the conduit retention device 230 may define a fixed attachment to the conduit. In still other embodiments, the conduit retention device 230 may be pre-attached to the pad 221.

In some embodiments, the conduit retention device 230 may be configured for attachment to an external apparatus, such as a bedrail, an IV pole, or the TTM module 210, for example. As such, one or more fluid conduits and/or the pad may be temporarily attached to the external apparatus to further define the storage configuration.

FIG. 5A illustrates the conduit retention device 230 including a strap 510 having one or more attachment features. The strap 510 defines a first end 511 and a second end 512 opposite the first end 511 and the strap 510 includes a first side 513 and a second side 514 opposite the first side 513.

The attachment features may include a loop 516. In some embodiments, the loop 516 may be disposed adjacent the first end 511. In other embodiments, the loop 516 may be disposed at any other location along the length of the strap 510 such as a center location, for example. The loop 516 may be sized to extend around a fluid conduit such as the fluid delivery conduit 221A, for example (see FIG. 2A). The loop 516 may be pre-threaded onto the fluid delivery conduit 221A during manufacture or the clinician may thread the loop 516 onto the fluid delivery conduit 221A at the patient care facility. In some embodiments, the loop 516 may be threaded onto the fluid return conduit 221B or fluid delivery conduit 221A and the fluid return conduit 221B. The loop 516 may be a fixed loop formed during manufacturing or the loop may be a non-fixed loop formed by the clinician at the patient care facility as described below. In the illustrated embodiment, the loop 516 may be slidably coupled to the fluid delivery conduit 221A allowing the clinician to position the conduit retention device 230 at any location along the length of the fluid delivery conduit 221A. In other embodiments, the loop 516 may be attached to the fluid delivery conduit 221A at a fixed location along the length of the fluid delivery conduit 221A.

The attachment features may further include a cinching ring 525. The cinching ring 525 may be disposed at the first end 511 as shown or at any other location spaced away from the second end 512. The cinching ring 525 may be configured so that during use of the conduit retention device 230, a portion of the strap 510 may be threaded through the cinching ring 525.

The strap 510 may include two or more complementary attachment components such as the first attachment component 517 and the second attachment component 518. The first and second attachment components 517, 518 may be configured to couple with each other. The attachment components 517, 518 may include a button and a hole, a snap, a buckle, a hook and loop fastener commonly referred to as “Velcro” or any other suitable attachment mechanism. The attachment component 518 may be positioned adjacent the second end 512 and the attachment component 517 may be spaced away from the second end 512. The first and second attachment components 517, 518 may be disposed on the first side 513 or on the first side 513 and the second side 514. In some embodiments, one or both of the first and second attachment components 517, 518 may extend along a substantial length of the strap 510 such as along 25 percent, 50 percent, 75 percent, or more of the length of the strap 510.

In some embodiments, the strap 510 may include attachment components in addition to the first and second attachment components 517, 518. For example, the strap 510 may include complementary attachment components 519, 520 configured to form a non-fixed loop 516. A non-fixed loop may provide placement of the loop 516 around a fluid conduit without threading the fluid conduit through the loop 516.

The strap 510 may be stretchable or non-stretchable. The strap 510 maybe formed of any suitable material including silicone, rubber, polyvinyl chloride (PVC), nylon, or a fabric such as cotton.

FIG. 5B illustrates an exemplary use case of the conduit retention device 230 of FIG. 5A. In some instances, the clinician may temporarily suspend the TTM therapy for the patient 50. In such instances, the clinician may disconnect the patient 50 from the TTM module 210 which may include removing one or more pads (e.g., the pad 221) of the pad set 220 from the patient 50 and/or disconnecting one or more pads of the pad set 220 from the TTM module 210. With the pad 221 separated from the patient 50, the clinician may dispose the pad 221 in a storage configuration as illustrated in FIG. 4C. As shown, the conduit retention devices 230A, 230B secure the fluid conduits 221A, 221B in the coiled configuration. As also shown, the band 470 secures the coiled fluid conduits 221A, 221B between the band 470 and the top side 401 of the pad portion 421. In some instances, it may be advantageous for the clinician to store the pad 221 at convenient location. FIG. 5B shows the pad 221 coupled to a bedrail 540. In the illustrated use case, the pad 221 is attached to the bedrail 540 via the conduit retention device 230B. In some use cases, the pad 221 may be stored as shown while the fluid conduits 221A, 221B remain coupled to the TTM module 210. In some use cases, the pad 221, when disposed in the storage configuration, may be coupled to the housing 211 of the TTM module 210 such as hung on a hook (not shown) of the TTM module 210. In other use cases, the pad 221 may be attached to any other suitable apparatus, such as an IV pole, for example.

FIG. 5C shows a cross-sectional view of the conduit retention device 230B according to the use case of FIG. 5B. As shown, the strap 510 is wrapped around the fluid conduits 221A, 221B thereby securing the fluid conduits 221A, 221B in the coiled state. The strap 510 is also wrapped around the bedrail 540 to secure the fluid conduits 221A, 221B to the bedrail 540. As is also shown, the fluid delivery conduit 221A is disposed within the loop 516 of the conduit retention device 230B. As also shown in FIG. 5C, the strap 510 is threaded through the cinching ring 525, and the second end 512 of the strap 510 is coupled with the strap 510 via the complementary attachment components 517, 518.

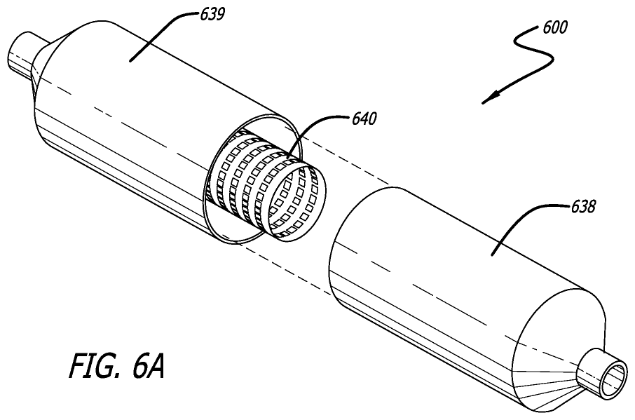

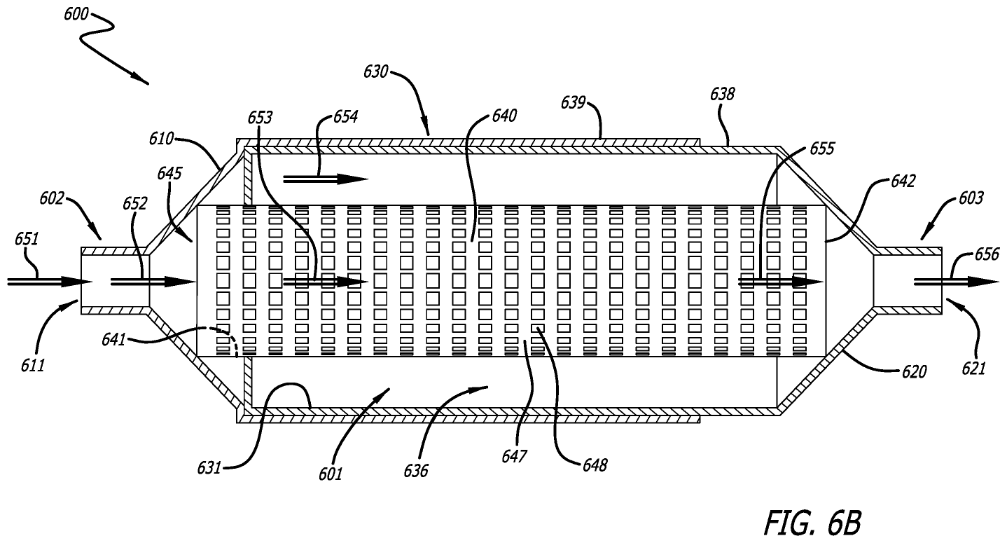

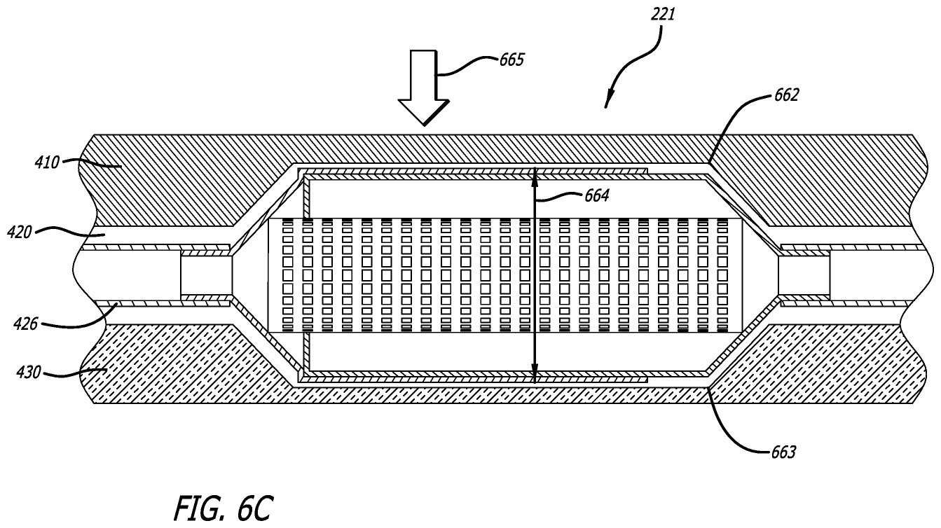

FIGS. 6A and 6B show a filter 600 that may be included with the TTM system 200. The filter 600 may be disposed in line with a TTM fluid flow path of the TTM system 200 so that the circulating TTM fluid 212 flows through the filter 600. The filter 600 may be configured to remove (i.e., filter out) material/particles having a size of 0.2 microns or larger from the TTM fluid 212 without causing a flow restriction of the TTM fluid 212.

The filter 600 includes a longitudinal shape having a flow path 601 extending from a first end 602 to a second end 603. The filter 600 includes a diffuser 610 adjacent the first end 602, a nozzle adjacent 620 the second end 603, and a body 630 extending between the diffuser 610 and the nozzle 620. Along the diffuser 610, a cross-sectional flow area of the filter 600 expands from an inlet flow area 611 to a body flow area 631 and along the nozzle 620, the cross-sectional flow area of the filter 600 contracts from the body flow area 631 to an outlet flow area 621. In some embodiments, the inlet flow area 611 and the outlet flow area 621 may be substantially equal.

In some embodiments, the body flow area 631 may be constant along the body 630. In other embodiments, the body flow area 631 may vary along a length of the body 630 such that the body flow area 631 is greater or less along middle portion of the body 630 than at the ends of the body 630. In some embodiments, the body flow area 631 may be circular.

The filter 600 includes an inner tube 640 disposed within the body 630 extending along the length of body 630. The inner tube 640 may be coupled with the diffuser 610 at a first inner tube end 641 so that TTM fluid 212 entering the filter 600 at the first end 602 also enters the inner tube 640 at the first inner tube end 641. The inner tube 640 may be coupled with the nozzle 620 at a second inner tube end 642 so that TTM fluid 212 exiting the filter 600 at the second end 603 also exits the inner tube 640 at the second inner tube end 642.

The inner tube 640 includes an inner tube flow area 645 extending the length of the inner tube 640. The inner tube flow area 645 may be greater than the inlet flow area 611 and/or the outlet flow area 621. The inner tube flow area 645 may be constant along the length of the inner tube 640. In some embodiments, the inner tube flow area 645 may vary along the length of the inner tube 640. In some embodiments, the inner tube 640 may include a circular cross section. The inner tube 640 and the body 630 may be configured so that the body flow area 631 includes a combination of the inner tube flow area 645 and an annular flow area 636.

The inner tube 640 includes a porous a circumferential wall 647. The porous wall 647 may be configured so that TTM fluid 212 may flow through the porous wall 647, i.e., through the pores 648 of the porous wall 647. Consequently, TTM fluid 212 may flow through the porous wall 647 from the inner tube flow area 645 to the annular flow area 636 and from the annular flow area 636 into the inner tube flow area 645.

In use, the longitudinal velocity of the TTM fluid 212 may change along the length of the filter 600. As the volumetric TTM fluid 212 flow through the filter is constant, the longitudinal velocity of the TTM fluid 212 may be at least partially defined by the flow areas of the filter 600 as described below. The TTM fluid 212 may enter the filter 600 at a first longitudinal velocity 651 and decrease along the diffuser so that the TTM fluid 212 enters the inner tube at a second velocity 652 less than the first longitudinal velocity 651. At this point, a portion of the TTM fluid 212 may flow through the porous wall 647 from the inner tube flow area 645 into the annular flow area 636 to divide the fluid flow into a third velocity 653 within the inner tube flow area 645 and a fourth velocity 654 within the annular flow area 636. The fourth velocity 654 may be less than the third velocity 653. A portion of the TTM fluid 212 may then flow back into the inner tube flow area 645 from the annular flow area 636 to define a fifth velocity 655 along the inner tube flow area 645 which may be about equal to the second velocity 652. The TTM fluid 212 may then proceed along the nozzle 620 to define a sixth velocity 656 exiting the filter 600. In some embodiments, the first velocity 651 and the sixth velocity 656 may be about equal.

The filter 600 may be configured to remove harmful bacteria and viruses from the TTM fluid 212 using sedimentation principles. In use, the filter 600 may be oriented horizontally so that the direction of fluid flow through the filter 600 is perpendicular to a gravitational force 665. In some instances, bacteria, viruses, and other particles within the TTM fluid 212 may have a greater density than the TTM fluid 212 and as such may be urged by the gravitational force 665 (i.e., sink) in a direction perpendicular to the fluid flow direction. In some instances, particles within the inner tube flow area 645 may sink toward and through the porous wall 647 into the annular flow area 636. Particles within the annular flow area 636 may then sink toward an inside surface 631 of the body 630 and become trapped adjacent the inside surface 631. The geometry of the filter 600 may be configured to allow 0.2-micron bacteria/virus particles to fall out of the flow of TTM fluid 212 and become trapped along the inside surface 631.

In some embodiments, the filter 600 may be configured so that flow of TTM fluid 212 from the inner tube flow area 645 into the annual flow area 636 my drag particles through the porous wall 647. In some embodiments, the inlet flow area 611, the inner tube flow area 645, and the annual flow area 636 may be sized so that the third velocity 653 is less than about 50 percent, 25 percent, or 10 percent of the first velocity 651 or less. In some embodiments, the body 630 and the inner tube 640 may be configured so that the fourth velocity 654 is less than the third velocity 653. In some embodiments, the fourth velocity 654 may less than about 50 percent, 25 percent, or 10 percent of the third velocity 653 or less.

In some embodiments, the filter 600 may be configured so that the flow within the inner tube flow area 645 is laminar flow, i.e., so that the velocity of the fluid flow adjacent to or in close proximity to an inside surface 641 of the porous wall 647 is less than the velocity at a location spaced away from the inside surface 641. In such an embodiment, the particles may more readily sink toward and through the porous wall 647.

In some embodiments, the filter 600 may be configured so that the fluid flow within the annual flow area 636 is laminar flow, i.e., so that the velocity of the fluid flow adjacent to or in close proximity to inside surface 631 of the body 630 is less than the velocity at a location spaced away from the inside surface 631. In such an embodiment, the particles may more readily sink toward and be trapped along the inside surface 631.

The filter 600 may include three components including the inner tube 640 an inner body shell 638, and an outer body shell 639. Each of the three components may be formed via the plastic injection molding process. Assembly of the filter 600 may include capturing the inner tube 640 within the inner body shell 638 and the outer body shell 639 and sliding the inner body shell 638 into the outer body shell 639 wherein the fit between the inner body shell 638 and the outer body shell 639 is an interference fit.

In some embodiments, the filter 600 may be disposed within a thermal pad such as the pad 221. FIG. 6C shows a detail cross-sectional view of the pad 221 including the filter 600 disposed within the fluid containing layer 420. The filter 600 is coupled in line with the internal fluid conduit 426 within the fluid containing layer 420 so that TTM fluid 212 circulating within the pad 221 passes through the filter 600. The filter 600 may be sized so that the inlet flow area 611 and the outlet flow area 621 are similar to a cross-sectional flow area of the internal flow path 426 within the fluid containing layer 420.

In some embodiments, a thickness of the fluid containing layer 420 may increase adjacent the filter 600 to accommodate a body diameter 664 of the filter 600. To further accommodate the body diameter 664, the insulation layer 410 and/or the thermal conduction layer 430 may include internal depressions 662, 663, respectively.

In some embodiments, one or more filters 600 may be disposed in line with the flow of TTM fluid 212 at other locations of the TTM system 200. In some embodiments, one or more filters 600 may be disposed within the TTM module 210. In some embodiments, one or more filters 600 may be disposed in line with the fluid conduits (e.g., the fluid delivery conduit 221A or the fluid return conduit 212B).

### Methods

Methods of the using the system may include the flowing steps or processes. The clinician may remove a thermal pad from a package, where the pad may be disposed in a shipping configuration. In the shipping configuration, the fluid conduits may be wound together to form a coil of windings. The windings may be bound together with a conduit retention device. The clinician may unwind the fluid conduits and extend the fluid conduits between the patient and the TTM module. The clinician may apply the pad to the patient. Thereafter, the TTM module may circulate fluid through the pad. The clinician may bind the TTM fluid delivery conduit and the fluid return conduits together with a conduit retention device.

The clinician may obtain a second thermal pad. The clinician may remove the second thermal pad from a package, where the second pad may be disposed in a shipping configuration as described above. The clinician may unwind the fluid conduits of the second thermal pad and extend the fluid conduits between the patient and the TTM module. The clinician may apply the second pad to the patient. Thereafter, the TTM module may circulate TTM fluid through the second pad. The clinician may bind the fluid delivery conduit and the fluid return conduits of the second pad together with a conduit retention device. The clinician may further bind one or both fluid conduits of the first pad together with one or both fluid conduits of the second pad with a conduit retention device. The clinician may further bind one or both fluid conduits of the first pad together with one or both fluid conduits of the second pad with two or more conduit retention devices.

The clinician may remove a thermal pad from the patient. The clinician may also disconnect the fluid conduits from the TTM module. The clinician may wind the fluid conduits together to form a coil and the bind two or more of windings of the coil together to maintain the coil. Thereafter, the clinician may place the coiled fluid conduits between the stretchable band and the top side of the pad to secure the coiled fluid conduits to the pad and thereby dispose the pad in a storage configuration. The clinician may also bind a bedrail together with the windings via a conduit retention device to secure the pad to the bedrail. In a similar manner, the clinician may remove, disconnect, and secure the second thermal pad.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a thermal pad configured to facilitate thermal energy transfer between the TTM fluid and a patient, the pad comprising:
a fluid delivery conduit extending continuously between the pad and the TTM module, the fluid delivery conduit configured to facilitate TTM fluid flow from the TTM module to the pad; and
a fluid return conduit extending continuously between the pad and the TTM module, the fluid return conduit configured to facilitate TTM fluid flow from the pad to the TTM module; and
a valve disposed in line with the fluid delivery conduit, the valve configured to selectively allow and prevent flow of TTM fluid through the fluid delivery conduit to the pad.

2. The system according to claim 1, wherein the valve is configured to automatically allow TTM fluid flow through the fluid delivery conduit upon connection of the respective fluid delivery conduit with the TTM module, and prevent TTM fluid flow through the fluid delivery conduit upon disconnection of the fluid delivery conduit from the TTM module.

3. The system according to claim 1, wherein the valve is configured for actuation by a processor of the TTM module in accordance with flow control logic of the TTM module to selectively allow and prevent TTM fluid flow through the fluid delivery conduit.

4. The system according to claim 1, wherein the fluid delivery conduit is coupled with the TTM module via an A-type connector attached to the fluid delivery conduit and a B-type connector attached to the TTM module.

5. The system according to claim 1, wherein the fluid return conduit is coupled with the TTM module via a B-type connector attached to the fluid return conduit and an A-type connector attached to the TTM module.

6. The system according to claim 4, wherein each A-type connector is configured to couple with and only with a B-type connector, and wherein each B-type connector is configured to couple with and only with an A-type connector.

7. The system according to claim 4, wherein each A-type connector includes a valve configured to automatically allow TTM fluid flow through the A-type connector upon connection of the A-type connector with a B-type connector and automatically prevent TTM fluid flow through the A-type connector upon disconnection of the A-type connector from the B-type connector.

8. The system according to claim 4, wherein each B-type connector includes a valve configured to automatically allow TTM fluid flow through the B-type connector upon connection of the B-type connector with an A-type connector and automatically prevent TTM fluid flow through the B-type connector upon disconnection of the B-type connector from the A-type connector.

9. The system according to claim 1, further comprising two or more conduit retention devices disposed along the fluid delivery conduit and/or the fluid return conduit, each conduit retention device configured for binding together the fluid delivery conduit and the fluid return conduit.

10. The system according to claim 9, wherein the thermal pad comprises at least one of the one or more conduit retention devices.

11. The system according to claim 10, wherein the at least one conduit retention device comprises a loop.

12. The system according to claim 11, wherein the loop is threaded onto the fluid delivery conduit or the fluid return conduit.

13. The system according to claim 11, wherein the loop is threaded onto the fluid delivery conduit together with the fluid return conduit.

14. The system according to claim 1, wherein the thermal pad comprises a stretchable band extending across a top side of the thermal pad, the stretchable band configured for disposing the thermal pad in a storage configuration.

15. The system according to claim 14, wherein the storage configuration comprises one or both of the fluid delivery conduit and the fluid return conduit disposed in a coiled configuration and placement of the coiled configuration between the stretchable band and the top side.

16. A medical pad for exchanging thermal energy between a targeted temperature management (TTM) fluid and a patient, the pad comprising:
a fluid containing layer, wherein:
the fluid containing layer is configured for containing the TTM fluid,
the fluid containing layer comprises a fluid inlet and a fluid outlet, and
the TTM fluid is circulatable within the fluid containing layer from the fluid inlet to the fluid outlet;
a fluid delivery conduit, wherein the fluid delivery conduit:
is coupled to the fluid inlet at a distal end,
is configured to extend continuously from the fluid containing layer to a TTM module, and
comprises a first A-type connector at a proximal end, the first A-type connector configured to couple with a first B-type connector disposed on a connection panel of the TTM module; and
a fluid return conduit, wherein the fluid return conduit
is coupled to the fluid outlet at a distal end,
is configured to extend continuously from the fluid containing layer and the TTM module, and
comprises a second B-type connector at a proximal end, the second B-type connector configured to couple with a second A-type connector disposed on the connection panel of the TTM module.

17. The medical pad according to claim 16, wherein each A-type connector is configured to couple with and only with a B-type connector, and wherein each B-type connector is configured to couple with and only with an A-type connector.

18. The medical pad according to claim 16, further comprising a conduit retention device configured to bind at least the fluid delivery conduit and the fluid return conduit together.

19. The medical pad according to claim 18, wherein the conduit retention device comprises a loop.

20. The medical pad according to claim 19, wherein the loop is threaded onto at least one of the fluid delivery conduit or the fluid return conduit.

21. The medical pad according to claim 19, wherein the loop is threaded onto the fluid delivery conduit and the fluid return conduit.

22. The medical pad according to claim 16, further comprising a stretchable band extending across a top side of the pad, the stretchable band configured for disposing the pad in a storage configuration.

23. The medical pad according to claim 22, wherein the storage configuration comprises one or both of the fluid delivery conduit and the fluid return conduit disposed in a coiled configuration and placed between the stretchable band and the top side.

24. The medical pad according to claim 16, wherein each A-type connector includes a valve configured to automatically allow TTM fluid flow through the A-type connector upon connection of the A-type connector with a B-type connector and automatically prevent TTM fluid flow through the A-type connector upon disconnection of the A-type connector from the B-type connector.

25. The medical pad according to claim 16, wherein each B-type connector includes a valve configured to automatically allow TTM fluid flow through the B-type connector upon connection of the B-type connector with an A-type connector and automatically prevent TTM fluid flow through the B-type connector upon disconnection of the B-type connector from the A-type connector.

26-33. (canceled)
